(19) United States
(12) Patent Application Publication
(10) Pub. No.: US 2026/0325721 A1
(43) Pub. Date: Oct. 22, 2026

(54) PROTECTIVE COVERINGS FOR DRUG COATED BALLOONS

(71) Applicant: BARD PERIPHERAL VASCULAR, INC., Franklin Lakes, NJ (US)

(72) Inventors: Terry A. Cruse, Lisle, IL (US); Katherine Huffer, Chandler, AZ (US); Brandon Simmons, Tempe, AZ (US)

(73) Assignee: BARD PERIPHERAL VASCULAR, INC., Franklin Lakes, NJ (US)

(21) Appl. No.: 19/486,753

(22) PCT Filed: May 22, 2023
(86) PCT No.: PCT/US2023/023106

§ 371 Date: Nov. 21, 2025

(51) Int. Cl. A61L 29/16; A61L 29/08; A61L 29/14

(52) U.S. Cl. CPC A61L 29/16; A61L 29/085; A61L 29/146; A61L 2420/08

(57) ABSTRACT

The present disclosure relates to protective layers over a drug-coating layer that restrict the expansion of the balloon. The protective layer is arranged so that parts of the balloon can expand through the protective layer and expose the coating layer and allow for transfer of the coating layer at the point of expansion. The protective layer has a higher modulus of elasticity that causes the balloon to expand through gaps therein.

### BACKGROUND

Drug coated balloon catheters provide site specific delivery of one or more therapeutics to assist the mechanical force within a blood vessel to attempt to clear obstructions therein. The coatings on the balloon need to be carefully crafted to allow for accurate transfer of the therapeutic(s) during the short residency time of the inflated balloon within the vessel. TO that end, the coatings on these devices are typically crafted to allow for quick release from the exterior surface of the balloon. The physical properties of these coatings, however, renders them vulnerable to the turbulence and fluid forces present within the vasculature as a user maneuvers the balloon to a desired location. Factors such as the length of time, distance from insertion to inflation, as well as the type of vessel and width of the vessel can all attribute to wash off of the coating before the balloon is even able to be inflated. Accordingly, there is a need in the art for protective coverings that allow the balloon to be positioned with any coating thereon in tact and deliver the therapeutic at the desired location.

### SUMMARY

A 1^{st} aspect of the present disclosure, either alone or in combination with any other aspect herein, concerns a balloon catheter for delivering a therapeutic agent to a blood vessel, the balloon catheter comprising: an elongate member having a lumen and a distal end; an expandable balloon attached to the distal end of the elongate member and in fluid communication with the lumen; a coating layer overlying an exterior surface of the expandable balloon, the coating layer comprising a therapeutic agent or a polymer microparticle containing the therapeutic agent and an excipient; and a protective layer overlying a portion of the coating layer, wherein the protective layer is of a material with higher elastic modulus than the expandable balloon.

A 2^{nd} aspect of the present disclosure, either alone or in combination with any other aspect herein, concerns the balloon catheter of the 1^{st} aspect, wherein the protective layer comprises at least one band about the circumference of the expandable balloon.

A 3^{rd} aspect of the present disclosure, either alone or in combination with any other aspect herein, concerns the balloon catheter of the 2^{nd} aspect, wherein the protective layer comprises at least two bands about the circumference of the expandable balloon.

A 4^{th} aspect of the present disclosure, either alone or in combination with any other aspect herein, concerns the balloon catheter of the 3^{rd} aspect, wherein each band is separated by a distance with exposed coating layer therebetween.

A 5^{th} aspect of the present disclosure, either alone or in combination with any other aspect herein, concerns the balloon catheter of the 3^{rd} aspect, wherein the bands overlap.

A 6^{th} aspect of the present disclosure, either alone or in combination with any other aspect herein, concerns the balloon catheter of the 1^{st} aspect, wherein the protective layer is a ribbon connected to a distal end and a proximal end of the expandable balloon and further wherein the ribbon wraps around the length of the balloon between the distal and proximal ends.

A 7^{th} aspect of the present disclosure, either alone or in combination with any other aspect herein, concerns the balloon catheter of the 6^{th} aspect, wherein the ribbon wraps at least once around the circumference of the balloon.

An 8^{th} aspect of the present disclosure, either alone or in combination with any other aspect herein, concerns the balloon catheter of the 7^{th} aspect, wherein the ribbon is wrapped such that the ribbon overlaps with itself as it progresses along the length of the expandable balloon.

A 9^{th} aspect of the present disclosure, either alone or in combination with any other aspect herein, concerns the balloon catheter of the 1^{st} aspect, wherein the protective layer has a modulus of elasticity of from 0.3 to about 2.5 GPa.

A 10^{th} aspect of the present disclosure, either alone or in combination with any other aspect herein, concerns the balloon catheter of the 1^{st} aspect, wherein the protective layer is comprised of polytetrafluoroethylene (PTFE), chlorotrifluoroethylene (E-CTFE), perfluoroalkoxy (PFA), or ethylene tetrafluoroethylene (ETFE).

An 11^{th} aspect of the present disclosure, either alone or in combination with any other aspect herein, concerns the balloon catheter of the 1^{st} aspect, wherein the protective layer has a thickness of from 100 μm to 600 μm.

A 12^{th} aspect of the present disclosure, either alone or in combination with any other aspect herein, concerns the balloon catheter of the 1^{st} or 11^{th} aspect, wherein the thickness of the protective layer varies.

A 13^{th} aspect of the present disclosure, either alone or in combination with any other aspect herein, concerns the balloon catheter of the 1^{st} aspect, wherein the coating layer comprises a therapeutic agent.

A 14^{th} aspect of the present disclosure, either alone or in combination with any other aspect herein, concerns the balloon catheter of the 13^{th} aspect, wherein the therapeutic agent is chosen from paclitaxel, rapamycin, daunorubicin, 5-fluorouracil, doxorubicin, sunitinib, sorafenib, irinotecan, bevasizumab, cetuxamab, biolimus (biolimus A9), everolimus, zotarolimus, tacrolimus, dexamethasone, prednisolone, corticosterone, cisplatin, vinblastine, lidocaine, bupivacaine, bosutinib, ceritinib, crizotinib, gefitinib, ruxolitinib, imatinib, axitinib, nilotinib, trametinib, afatinib, ibrutinib, cabozantinib, imatinib, lenvatinib, sunitinib, regorafenib, sorafenib, vandetanib, dasatinib, pazopanib, triamciclone, tranilast, halofuginone, montelukast, zafirlukast, pirfenidone, Y27632, CA3, verteporfin, VGLL4 peptide, nintedanib, avapritinib, abemaciclib, erdafitinib, fedratinib, nilotinib, nintendanib, palbociclib, pemigatinib, xanthines, aminophylline, sildenafil, tadalafil, vardenafil, udenafil, avanafil, dipyridamole, quinazoline, paraxanthine, papaverine, mesembrenone, rolipram, ibudilast, piclamilast, luteolin, drotaverine, roflumilast, apremilast, crisaborole, inamrinone, milrinone, enoximone, anagrelide, cilostazol, pimobendan, erythro-9-(2-hydroxy-3-nonyl)adenine), (2-[(3,4-dimethoxyphenyl)methyl]-7-[(1R)-1-hydroxyethyl]-4-phenylbutyl]-5-methyl-imidazo[5,1-f][1,2,4]triazin-4(1H)-one), oxindole, (9-(6-phenyl-2-oxohex-3-yl)-2-(3,4-dimethoxybenzyl)-purin-6-one), 3-isobutyl-1-methylxanthine, pentoxifylline, theobromine, theophylline, resveratrol, quercetin, curcumin, chrysin, myricetin, luteolin, apigenin, anthrocyanin, genistein, epigallocatechin gallate, fisetin, astxanthin, tetrahydrocurcumin, imatinib, nintedanib, sorafenib, sunitinib, pazopanib, ROCK inhibitor (Y27632), YAP/TAZ inhibitor (CA3 and verteporfin), YAP/TAZ-TEAD interaction inhibitor (verteporfin, VGLL4 peptide), SRC inhibitor (dasatinib), and combinations thereof.

A 15^{th} aspect of the present disclosure, either alone or in combination with any other aspect herein, concerns the balloon catheter of the 13^{th} aspect, wherein the coating layer further comprises a biodegradable polymer.

A 16^{th} aspect of the present disclosure, either alone or in combination with any other aspect herein, concerns the balloon catheter of the 15^{th} aspect, wherein the biodegradable polymer is chosen from a polylactic acid polymer, polycaprolactone (PCL), poly lactic-co-glycolic acid (PLGA), and poly(ethylene glycol) methyl ether-block-poly(lactide-co-glycolide) (PLGA-b-mPEG).

A 17^{th} aspect of the present disclosure, either alone or in combination with any other aspect herein, concerns the balloon catheter of the 13^{th} or 15^{th} aspect, wherein the coating layer further comprises an excipient chosen from a fatty acid, a fatty acid ester, polylactic acid (PLLA, PDLA, PDLLA), polycaprolactone (PCL), sodium docusate, PLGA, PLGA-b-mPEG, polyglutamic acid, polyacrilic acid, hyaluronic acid, alginate, PVA, PVP, Pluronic (PEO-PPO-PEO), cellulose, CMC, HPC, starch, chitosan, human serum albumin (HSA), phospholipids, fatty acid, fatty acid esters, triglycerides, beeswax, cyclodextrin, Tween 20, Tween 80, TPGS, SLS, butylated hydroxytoluene, vitamin E, vitamin E succinate, tannic acid, polyethylene glycol, N-isopropylacrylamide, and sorbitol esters.

An 18^{th} aspect of the present disclosure, either alone or in combination with any other aspect herein, concerns the balloon catheter of the 13^{th}, 15^{th}, or 17^{th} aspect, wherein the coating layer further comprises a hydrophobic material containing the therapeutic agent embedded therein.

A 19^{th} aspect of the present disclosure, either alone or in combination with any other aspect herein, concerns the balloon catheter of the 18^{th} aspect, wherein the hydrophobic material comprises a hydrophobic material with a glass transition temperature of 37 °C or lower.

A 20^{th} aspect of the present disclosure, either alone or in combination with any other aspect herein, concerns the balloon catheter of the 18^{th} aspect, wherein the hydrophobic material is semi-synthetic glycerides, methyl stearate, hydrogenated coconut oil, coconut oil, cocoa butter, glycerinated gelatin, hydrogenated vegetable oils, hard fats, petroleum jelly/petrolatum, a PEG-fatty acid ester, or a combination thereof.

A 21^{st} aspect of the present disclosure, either alone or in combination with any other aspect herein, concerns the balloon catheter of the 20^{th} aspect, wherein the hydrophobic material is hydrogenated coconut oil, coconut oil, mineral oil, cetyl alcohol, petroleum jelly, decanol, tridecanol, dodecanol, long chain saturated fatty acids, long chain unsaturated fatty acid, fatty acid esters, fatty acid ethers, witepsol, solid lipids, methyl stearate, triglycerides, glyceryl monostearate, glyceryl palmitostearate, stearic acid, palmitic acid, decanoic acid, behenic acid, beeswax, carnauba wax, paraffin, a fatty acid triglycerides, a fatty acid alcohol, or a combination thereof.

A 22^{nd} aspect of the present disclosure, either alone or in combination with any other aspect herein, concerns the balloon catheter of the 13^{th}, 15^{th}, or 17^{th} aspect, wherein the therapeutic agent is contained within a polymer microparticle.

A 23^{rd} aspect of the present disclosure, either alone or in combination with any other aspect herein, concerns the balloon catheter of the 22^{nd} aspect, wherein the polymer microparticle comprises poly(lactic-co-glycolic) acid (PLGA) and the therapeutic agent loaded therein.

A 24^{th} aspect of the present disclosure, either alone or in combination with any other aspect herein, concerns the balloon catheter of the 23^{rd} aspect, wherein the therapeutic agent is loaded in the polymer microparticle at 30-50 % weight of the polymer microparticle.

A 25^{th} aspect of the present disclosure, either alone or in combination with any other aspect herein, concerns the balloon catheter of the 24^{th} aspect, wherein the polymer microparticles are of a first size grouping and a second size grouping, wherein the first size grouping has an average size of 10 μm and further wherein the second size grouping has an average size different from the first size grouping.

A 26^{th} aspect of the present disclosure, either alone or in combination with any other aspect herein, concerns the balloon catheter of the 25^{th} aspect, wherein the second size grouping has an average size of 30 μm, 35 μm, or 40 μm.

A 27^{th} aspect of the present disclosure, either alone or in combination with any other aspect herein, concerns the balloon catheter of the 13^{th}, 15^{th}, or 17^{th} aspect, wherein the therapeutic agent is crystalline particles.

A 28^{th} aspect of the present disclosure, either alone or in combination with any other aspect herein, concerns the balloon catheter of the 27^{th} aspect, wherein the average size of the crystalline particles is of 0.1 μm to 100 μm.

A 29^{th} aspect of the present disclosure, either alone or in combination with any other aspect herein, concerns the balloon catheter of the 13^{th}, 15^{th}, or 17^{th} aspect, wherein the coating layer further comprises a hydrophilic material chosen from poly(ethylene glycol), polyvinyl pyrrolidone, polyvinyl alcohol, polyacrylic acid, polyacrylamides, N-(2-Hydroxypropyl) methacrylamide (HPMA), divinyl ether-maleic anhydride (DIVEMA), polyoxazoline, xanthan gum, pectins, chitosan derivatives, dextran, casein sodium, cellulose ethers, sodium carboxy methyl cellulose, hydroxypropylmethyl cellulose (HPMC), hydroxypropyl cellulose (HPC), hydroxyethyl cellulose (HEC), hyaluronic acid (HA), albumin, or a combination thereof.

A 30^{th} aspect of the present disclosure, either alone or in combination with any other aspect herein, concerns a method for treating a blood vessel in a subject comprising introducing the balloon catheter of the 1^{st} aspect into the blood vessel of the subject; maneuvering the balloon catheter to a atherosclerotic plaque in the blood vessel; and, expanding the expandable balloon, wherein the protective coating dissolves prior to expanding the expandable balloon.

A 31^{st} aspect of the present disclosure, either alone or in combination with any other aspect herein, concerns a balloon catheter for delivering a therapeutic agent to a blood vessel, the balloon catheter comprising: an elongate member having a lumen and a distal end; an expandable balloon attached to the distal end of the elongate member and in fluid communication with the lumen; a porous layer overlying an exterior surface of the expandable balloon, the porous layer comprising a therapeutic agent or a polymer microparticle containing the therapeutic agent and an excipient; and a protective layer overlying all or at least 50% of the coating layer when the expandable balloon in a folded, deflated position, wherein the protective layer is of a material with higher elastic modulus than the expandable balloon.

A 32^{nd} aspect of the present disclosure, either alone or in combination with any other aspect herein, concerns the balloon catheter of the 31^{st} aspect, wherein the protective layer comprises at least one band about the circumference of the expandable balloon.

A 33^{rd} aspect of the present disclosure, either alone or in combination with any other aspect herein, concerns the balloon catheter of the 32^{nd} aspect, wherein the protective layer comprises at least two bands about the circumference of the expandable balloon.

A 34^{th} aspect of the present disclosure, either alone or in combination with any other aspect herein, concerns the balloon catheter of the 33^{rd} aspect, wherein each band is separated by a distance with exposed coating layer therebetween.

A 35^{th} aspect of the present disclosure, either alone or in combination with any other aspect herein, concerns the balloon catheter of the 33^{rd} aspect, wherein the bands are positioned in a vertical, angled, helical or combination thereof arrangement about the exterior surface of the balloon.

### BRIEF DESCRIPTION OF THE DRAWINGS

It is to be understood that both the foregoing general description and the following detailed description describe various aspects and are intended to provide an overview or framework for understanding the nature and character of the claimed subject matter. The accompanying drawings are included to provide a further understanding of the various aspects, and are incorporated into and constitute a part of this specification. The drawings illustrate the various aspects described herein, and together with the description serve to explain the principles and operations of the claimed subject matter.

FIG. 1 is a schematic of an exemplary aspect of a medical device, particularly a balloon catheter, according to the present disclosure.

FIG. 2A is a cross-section of some aspect of the distal portion of the balloon catheter of FIG. 1, taken along line A-A, including a drug coating layer on an exterior surface of a balloon.

FIG. 2B and is a cross-section of some aspect of the distal portion of the balloon catheter of FIG. 1, taken along line A-A, including an intermediate layer between a exterior surface of the balloon and a drug coating layer.

FIG. 3A is a sideview of some aspect of the balloon enlarged from FIG. 1 including a protective layer around the balloon.

FIG. 3B is a sideview of some aspect of the balloon enlarged from FIG. 1 including a protective layer around the balloon.

### DESCRIPTION

The present disclosure pertains to protective layer(s) that cover, at least in part, the drug coating on the exterior surface of a balloon, In some aspects, the presence of the protective layer shields the drug coating from the turbulence of a subjects circulatory system, as well as against abrasion along the vessel wall as the balloon is maneuvered within the subject to a desired location. It is an aspect of the present disclosure that once in place in situ, the balloon will inflate and release or transfer the drug coating at the site of inflation.

The protective layer is a series of bands or a spiraled ribbon that proceed along the length of a balloon between the proximal and distal ends thereof. The spiraled ribbon may be secured to the distal and proximal ends of the balloon or to the underlying catheter or guidewire. In some aspects, the bands or spiraled ribbon may wrapped around the circumference of the exterior surface of the balloon without an adhesive or point of fusion or adherence to the exterior surface of the balloon or a drug coating thereon. In other aspects, the bands or spiraled ribbon may be adhered to the exterior surface od the balloon at one or more points or lines of adhesion. It will be appreciated that as described herein, the balloon moves around the protective layer as inflation occurs. Accordingly, the bands or spiraled ribbon should not be fused or adhered to the balloon in a manner that restricts the balloon from inflating around the protective layer. The bands or spiraled ribbon may be adhered through a weld, an adhesive, a suture, or similar that allows for the band or ribbon to be secured to the balloon without negatively impacting the ability of the balloon to inflate or retain air once inflated.

In aspects, the bands or spiraled ribbon are arranged such that they cover the length of the balloon from the proximal to the distal end when not inflated. In some aspects, the bands or ribbon may overlap slightly. It will be appreciated that a significant overlap may negatively impede the ability of the balloon to move around the protective layer as the balloon attempts to inflate. In aspects, the bands or spirals are spaced such that the underlying balloon or coating thereon is visible therebetween. It will be appreciated that the larger spacing between bands or spiral repeats allows for increased expansion of the underlying balloon.

In aspects, the bands or spiraled ribbon are of a material with a different elasticity modulus than that of the balloon. In aspects, the material of the bands or spiraled ribbon are less elastic than the balloon or more resistant to expanding. As such, as the balloon inflates, the material of the balloon expands past the protective layer. As the balloon expands, the bands or spiraled ribbon may shift, such as along the length of the balloon and/or fold in on itself or bunch together, but will not expand in unison or at the same rate as the balloon. As such, the force of the inflation allows the exterior surface of the balloon to move around the bands or between the wraps of the spiraled ribbon.

In aspects, the balloon is of a material that may include a polymer material, such as, for example only, polyvinyl chloride (PVC), polyethylene terephthalate (PET), polyethylene, Nylon, PEBAX (i.e. a copolymer of polyether and polyamide), polyurethane, polystyrene (PS), polyethleneterephthalate (PETP), or various other suitable materials with a similar elastic modulus.

In aspects, the protective layer is of a material with a higher elastic modulus or more resistant to expansion. As such, as the underlying balloon expands, the protective layer remains relatively in place with regard to expansion and the balloon is forced to expand through gaps or spaces between the protective layer. For example, a protective layer of polytetrafluoroethylene (PTFE) has a high elastic modulus and is more resistant to the underlying expansion of the balloon. In some aspects, the material may be, but is not limited to, a PTFE substitute such as expanded polytetrafluoroethylene (ePTFE), chlorotrifluoroethylene (E-CTFE), perfluoroalkoxy (PFA), ethylene tetrafluoroethylene (ETFE) In some aspects, the material has a modulus of elasticity of from about 0.3 to about 2.5 GPa, including about 0.4, 0.5, 0.6, 0.7, 0.8, 0.9, 1.0, 1.1, 1.2, 1.3, 1.4, 1.5,1.6, 1.7, 1.8, 1.9, 2.0, 2.1, 2.2, 2.3, and 2.4 GPa. It will also be appreciated that the material of the protective layer provides a low coefficient of friction, thereby allowing the balloon greater freedom of movement within the subject's vessel lumen.

In some aspects, the protective layer may be scored or expanded, thereby allowing the protective layer to thin and expose the underlying balloon as the balloon expands. In such aspects, the protective layer may possess a modulus of elasticity that is less that that of the material of the underlying balloon.

In some aspects, the thickness of the protective layer may serve to increase the resistance to expansion. In some aspects, the protective layer is of a thickness of from about 100 to 600 um in thickness, including about 125, 150, 175, 200, 225, 250, 275, 300, 325, 350, 375, 400, 425, 450, 475, 500, 525, 550, and 575 μm thick. It will also be appreciated that the thickness need not be uniform. In some aspects, introducing thinner regions or zones in the material will reduce the modulus of elasticity at those points and allow the underlying expandable balloon easier access to push past at those regions. Accordingly, in some aspects, the material may vary in thickness by from about 50 to about 250 μm (depending on the original thickness), including about 75, 100, 125, 150, 175, 200, and 225 μm.

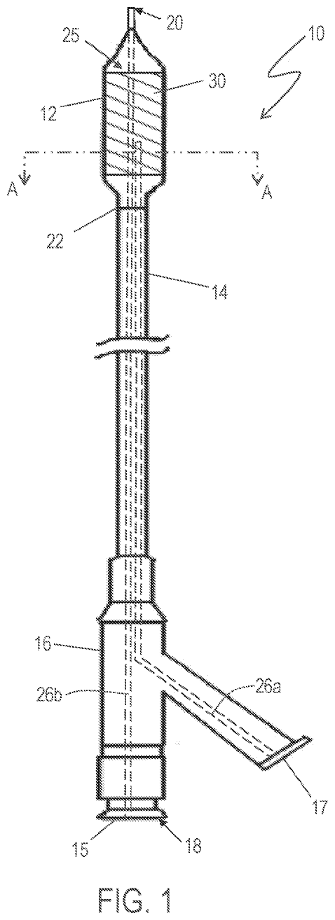

Referring to the exemplary drawing of FIG. 1, a balloon catheter 10 has a proximal end 18 and a distal end 20. The balloon catheter 10 may be any suitable catheter for desired use, including conventional balloon catheters known to one of ordinary skill in the art. For example, the balloon catheter 10 may be a rapid exchange or over-the-wire catheter. In some specific examples, the balloon catheter may be a ClearStream™ Peripheral catheter available from BD Peripheral Intervention. The balloon catheter 10 may be made of any suitable biocompatible material. The balloon 12 of the balloon catheter may include a polymer material, such as, for example only, polyvinyl chloride (PVC), polyethylene terephthalate (PET), polyethylene, Nylon, PEBAX (i.e. a copolymer of polyether and polyamide), polyurethane, polystyrene (PS), polyethleneterephthalate (PETP), or various other suitable materials as will be apparent to those of ordinary skill in the art.

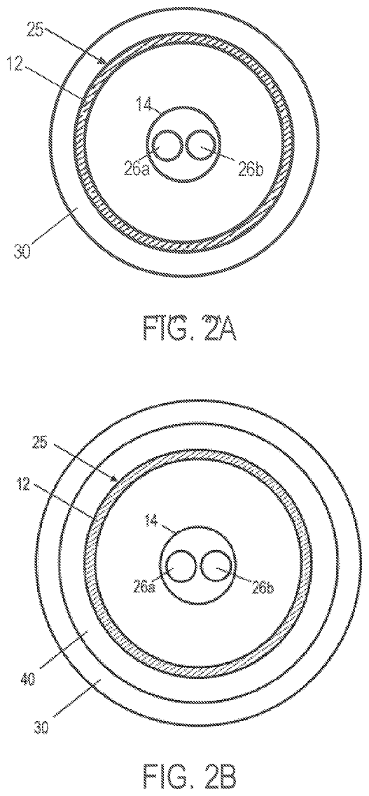

Referring jointly to FIGS. 1, 2A, and 2B, the balloon catheter 10 includes an expandable balloon 12 and an elongate member 14. The elongate member 14 extends between the proximal end 18 and the distal end 20 of the balloon catheter 10. The elongate member 14 has at least one lumen 26a, 26b and a distal end 20. The elongate member 14 may be a flexible member which is a tube made of suitable biocompatible material. The elongate member 14 may have one lumen or, as shown in FIGS. 1, 2A, and 2B, more than one lumen 26a, 26b therein. For example, the elongate member 14 may include a guide-wire lumen 26b that extends to the distal end 20 of the balloon catheter 10 from a guide-wire port 15 at the proximal end 18 of the balloon catheter 10. The elongate member 14 may also include an inflation lumen 26a that extends from an inflation port 17 of the balloon catheter 10 to the inside of the expandable balloon 12 to enable inflation of the expandable balloon 12. From the elements of FIGS. 1, 2A, and 2B, even though the inflation lumen 26a and the guide-wire lumen 26b are shown as side-by-side lumens, it should be understood that the one or more lumens present in the elongate member 14 may be configured in any manner suited to the intended purposes of the lumens including, for example, introducing inflation media and/or introducing a guide-wire. Many such configurations are well known in the art.

The expandable balloon 12 is attached to the distal attachment end 22 of the elongate member 14. The expandable balloon 12 has an exterior surface 25 and is inflatable. The expandable balloon 12 is in fluidic communication with a lumen of the elongate member 14, (for example, with the inflation lumen 26a). At least one lumen of the elongate member 14 is configured to receive inflation media and to pass such media to the expandable balloon 12 for its expansion. Examples of inflation media include air, saline, and contrast media.

Still referring to FIG. 1, in one aspect, the balloon catheter 10 includes a handle assembly such as a hub 16. The hub 16 may be attached to the balloon catheter 10 at the proximal end 18 of the balloon catheter 10. The hub 16 may connect to and/or receive one or more suitable medical devices, such as a source of inflation media (e.g., air, saline, or contrast media) or a guide wire. For example, a source of inflation media (not shown) may connect to the inflation port 17 of the hub 16 (for example, through the inflation lumen 26a), and a guide wire (not shown) may be introduced to the guide-wire port 15 of the hub 16, (for example through the guide-wire lumen 26b).

In some examples, the cross section A-A of FIG. 1 may be as depicted according to FIG. 2A, in which the drug coating layer 30 is applied directly onto an exterior surface 25 of the balloon 12. The specific compositions of the drug coating layer 30 itself, according to various aspects, will also be described subsequently in greater detail. In other examples, the cross section A-A of FIG. 1 may be as depicted according to FIG. 2B, in which the drug coating layer 30 is applied onto an intermediate layer 40 overlying the exterior surface 25 of the balloon 12. In some aspects, the exterior surface 25 may undergo a surface modification. In some aspects where the exterior surface 25 is a modified exterior surface, the exterior surface 25 has been subjected to a surface modification, such as a fluorine plasma treatment, which decreases a surface free energy of the exterior surface 25 before application of the drug coating layer 30. Subjecting the exterior surface to a surface modification may decreases the surface free energy of the exterior surface before application of the coating layer and affect the release kinetics of drug in the coating layer from the balloon, the crystallinity of the drug layer, the surface morphology of the coating and particle shape, or the particle size of drug of a therapeutic layer in the coating layer, drug distribution on the surface.

In aspects in which the cross section A-A of FIG. 1 is as depicted according to FIG. 2A, the balloon catheter 10 includes a drug coating layer 30 applied over an exterior surface 25 of the balloon 12. The drug coating layer 30 itself includes a therapeutic agent and an additive. In one particular aspect, the drug coating layer 30 comprises a kinase inhibitor, tyrosine kinase inhibitor, a PDE inhibitor, or an anti-fibrotic therapeutic agent, the polymer, and one or more additional additives. In further aspects, the drug coating layer 30 does not include a polymer.

In other aspects, two or more therapeutic agents are used in combination in the drug coating layer. In other aspects, the device may include a top layer (not shown) overlying the drug coating layer 30. In some aspects, a protective layer 55 may be advantageous in order to prevent premature drug loss during the device delivery process before deployment at the target site.

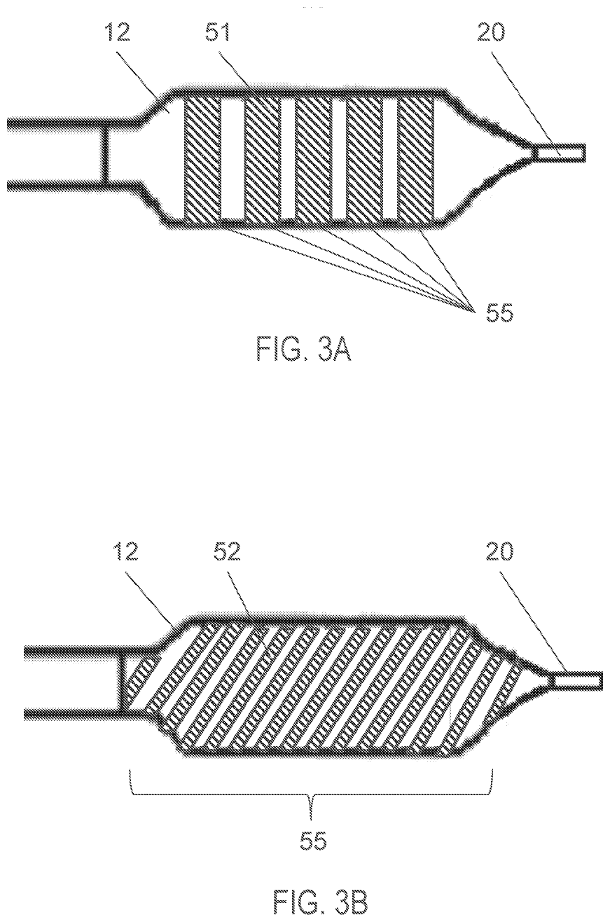

Referring to FIG. 3A, the exterior surface of the expandable balloon 12 is depicted in one aspect of the present disclosure, wherein several bands 51 are positioned along the length of the expandable balloon 12. As depicted, the bands 51 form a protective layer 55 that covers at least a part of the exterior surface of the expandable balloon 12. It will be appreciated that the bands 51 can be spaced such that they overlap, touch, or have exposed expandable balloon 12 in between.

Referring to FIG. 3B, the exterior surface of the expandable balloon 12 is depicted in a further aspect of the present disclosure. The protective layer 55 is formed from the spiraling repeats of the ribbon 52. The spirals of the ribbon 52 are positioned along the length of the expandable balloon 12. As depicted, the ribbon 52 forms a protective layer 55 that covers at least a part of the exterior surface of the expandable balloon 12. It will be appreciated that the spirals of the ribbon 52 can be spaced such that they overlap, touch, or have exposed expandable balloon 12 in between.

In aspects, the expandable balloon includes an intermediate porous layer between the exterior surface of the balloon and the protective layer. It will be appreciated that due to the lower elastic modulus of the protective layer, an intermediate porous layer will receive pressure from the protective layer and the exterior surface of the balloon as the balloon expands. It is therefore an aspect of the present disclosure that loading a porous intermediate layer with a therapeutic or a therapeutic containing solution will release during balloon expansion due to the concerted pressure. Similarly, providing patterned bands (such as with vertical bands, helical bands, angled bands or combinations thereof about the balloon) as the protective layer will allow for a similar compression of the intermediate porous layer as the balloon expands. It will be apparent that in such arrangements, the coating layer is not required or is loaded within the porous material.

In aspects, the protective layer covers at least in part a coating layer on the exterior surface of the balloon. In aspects, the coating layer includes one or more therapeutic agents. In some aspects, the therapeutic agent is chosen from paclitaxel, rapamycin, daunorubicin, 5-fluorouracil, doxorubicin, sunitinib, sorafenib, irinotecan, bevasizumab, cetuxamab, biolimus (biolimus A9), everolimus, zotarolimus, tacrolimus, dexamethasone, prednisolone, corticosterone, cisplatin, vinblastine, lidocaine, bupivacaine, bosutinib, ceritinib, crizotinib, gefitinib, ruxolitinib, imatinib, axitinib, nilotinib, trametinib, afatinib, ibrutinib, cabozantinib, imatinib, lenvatinib, sunitinib, regorafenib, sorafenib, vandetanib, dasatinib, pazopanib, triamciclone, tranilast, halofuginone, montelukast, zafirlukast, pirfenidone, Y27632, CA3, verteporfin, VGLL4 peptide, nintedanib, avapritinib, abemaciclib, erdafitinib, fedratinib, nilotinib, nintendanib, palbociclib, pemigatinib, xanthines, aminophylline, sildenafil, tadalafil, vardenafil, udenafil, avanafil, dipyridamole, quinazoline, paraxanthine, papaverine, mesembrenone, rolipram, ibudilast, piclamilast, luteolin, drotaverine, roflumilast, apremilast, crisaborole, inamrinone, milrinone, enoximone, anagrelide, cilostazol, pimobendan, erythro-9-(2-hydroxy-3-nonyl)adenine), (2-[(3,4-dimethoxyphenyl)methyl]-7-[(1R)-1-hydroxyethyl]-4-phenylbutyl]-5-methyl-imidazo[5,1-f][1,2,4]triazin-4(1H)-one), oxindole, (9-(6-phenyl-2-oxohex-3-yl)-2-(3,4-dimethoxybenzyl)-purin-6-one), 3-isobutyl-1-methylxanthine, pentoxifylline, theobromine, theophylline, resveratrol, quercetin, curcumin, chrysin, myricetin, luteolin, apigenin, anthrocyanin, genistein, epigallocatechin gallate, fisetin, astxanthin, tetrahydrocurcumin, imatinib, nintedanib, sorafenib, sunitinib, pazopanib, ROCK inhibitor (Y27632), YAP/TAZ inhibitor (CA3 and verteporfin), YAP/TAZ-TEAD interaction inhibitor (verteporfin, VGLL4 peptide), SRC inhibitor (dasatinib), and combinations thereof.

In some aspects, the coating layer may include microparticles of therapeutic agent(s) and a bioabsorbable polymer. Microparticles may be prepared the evaporation of a solvent with a bioabsorbable/biodegradable polymer and at least one therapeutic therein. In some aspects, the solvent is of dichloromethane (DCM) or ethyl acetate (EtOAc). Polymers may include a network of a poly-glycolic acid (PGA) and a poly-L-lactic acid (PLLA). Other bioabsorbable polymers that can be utilized in combination or alone for the microparticles include polycaprolactone (PCL), poly-DL-lactic acid (PDLLA), poly(trimethylene carbonate) (PTMC), poly (ester amine)s (PEA), poly(para-dioxanone) (PPDO), poly-2-hydroxy butyrate (PHB), and co-polymers with various ratios thereof. In some aspects, the bioabsorbable polymer may include, either alone or in combination with other bioabsorbable polymers, a polymer combination of lactic acid and glycolic acid, poly-lactic-co-glycolic acid (PLGA). Those skilled in the art will appreciate that PLGA can be of varying percentages of lactic acid and glycolic acid, wherein the higher the amount of lactide units, the longer the polymer can last in situ before degrading. Additional tunable properties with PLGA concern the molecular weight, with higher weights showing increased mechanical strength. In some aspects, the polymer microparticle is also loaded or embedded with an antioxidant, such as BHT.

In some aspects, the coating layer may include a polymer coating, such as a bioabsorbable polymer as set forth herein. In some aspects, the density of the therapeutic or polymer microparticle within the polymer coating is of from about 0.1 to 10 μg/mm². In certain aspects, the theapeutic or polymer microparticle is provided on the device in the polymer coating at a density of from about 0.5 to about 5 μg/mm². In some aspects, the dose density of the therapeutic in the coating and/or within each polymer microparticle can vary from about 0.1 to about 10 μg/mm², including about 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8., 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, and 9.9 μg/mm². In some aspects, the therapeutic dose density is of about 0.5 to about 5 μg/mm².

In some aspects, the concentration density of the therapeutic agent in the coating layer or within the polymer microparticle may be from 0.1 μg/mm² to 10 μg/mm², from 0.1 μg/mm² to 8 μg/mm², from 0.1 μg/mm² to 6 μg/mm², from 0.1 μg/mm² to 4 μg/mm², from 0.1 μg/mm² to 2 μg/mm², from 0.1 μg/mm² to 1 μg/mm², from 1 μg/mm² to 10 μg/mm², from 1 μg/mm² to 8 μg/mm², from 1 μg/mm² to 6 μg/mm² , from 1 μg/mm² to 4 μg/mm², from 1 μg/mm² to 2 μg/mm², from 2 μg/mm² to 10 μg/mm², from 2 μg/mm² to 8 μg/mm², from 2 μg/mm² to 6 μg/mm², from 2 μg/mm² to 4 μg/mm², from 4 μg/mm² to 10 μg/mm², from 4 μg/mm² to 8 μg/mm², from 4 μg/mm² to 6 μg/mm², from 6 μg/mm² to 10 μg/mm², from 6 μg/mm² to 8 μg/mm², or from 8 μg/mm² to 10 μg/mm². In some aspects the concentration density of therapeutic agent in the coating layer or polymer microparticle may be from 0.5 μg/mm² to 5 μg/mm².

The methods to apply the coating layer include to a medical device may include dip coating, metering coating, spray coating, electrostatic spray coating, roller coating, spin coating, ink-jet printing, 3D printing, or combinations thereof. A preferred method is metering coating and spray coating. After the solvent has evaporated, the coating layer is left on the surface.

In some aspects, the coating layer may include crystalline therapeutic agent and/or an amorphous therapeutic agent of a particular size range or ranges. In some aspects, the crystalline and/or amorphous therapeutic agent can be embedded within the coating layer. In other aspects, the crystalline and/or amorphous therapeutic agent is loaded within a polymer microparticle embedded in the coating layer. In further aspects, the crystalline and/or amorphous therapeutic agent adheres to the surface of the medical device through the evaporation of a solvent. In some aspects, the crystalline and/or amorphous therapeutic agent microparticle size can vary from about 0.1 μm to about 100 μm, including about 0.2, 0.3, 0.4, 0.5, 0.6, 0.7, 0.8, 0.9, 1.0, 2, 3, 4, 5, 6, 7, 8, 9, 10, 20, 30, 40, 50, 60, 70, 80, 90, 99 um and any size or number therein. In some aspects, the particle size is of from about 1 μm to about 20 μm. In other aspects, the particle size of from about 10 μm to about 100 μm. Size selection can be achieved through methods understood in the art, such as by passing through mesh of a pre-determined pore or hole size. The desired particle size can be achieved by dry grind, microfluidics, or wet grinding. The grinding method may include techniques such as use of a jaw crusher, ultra-centrifugal mill, cyclone mill, cross beater mill, rotor beater mill, cutting mill, knife mill, mortar grinder, disc mill, mixer mill, cryomill, planetary ball mill, drum mill, and/or fine grinding rod mill. In some aspects, the particle size may be achieved with use of a ball mill. The ground drug particles and polymer mix may be combined with a solvent (or a mixture of solvents) and form a slurry coating solution. The methods may also include application of the slurry coating solution to a medical device surface. Such techniques for application may include dip coating, metering coating, spray coating, electrostatic spray coating, roller coating, spin coating, ink-jet printing, and 3D printing. In certain aspects, the method includes metering coating.

In aspects, the coating layer includes a hydrophobic polymer with the therapeutic dispersed throughout. In some aspects, the biodegradable polymer chosen from a fatty acid, a fatty acid ester, polylactic acid (PLLA, PDLA, PDLLA), polycaprolactone (PCL), sodium docusate, PLGA, PLGA-b-mPEG, polyglutamic acid, polyacrilic acid, hyaluronic acid, alginate, PVA, PVP, Pluronic (PEO-PPO-PEO), cellulose, CMC, HPC, starch, chitosan, human serum albumin (HSA), phospholipids, fatty acid, fatty acid esters, triglycerides, beeswax, cyclodextrin, Tween 20, Tween 80, TPGS, SLS, butylated hydroxytoluene, vitamin E, vitamin E succinate, tannic acid, polyethylene glycol, N-isopropylacrylamide, and sorbitol esters. In further aspects, the polymer is a bioabsorbable polymer of a hydrophobic or hydrophilic nature. By way of example and not limitation, examples of bioabsorbable hydrophobic materials may include semi-synthetic glycerides (e.g. Suppocire AIML, AML, BML, BS2, BS2X, NBL, NAIS 10, CS2X), lecithin, hydrogenated coconut oil, coconut oil, cocoa butter, glycerinated gelatin, hydrogenated vegetable oils, hard fats, mineral oil, cetyl alcohol, petrolatum, petroleum jelly, decanol, soft paraffin, tridecanol, dodecanol, long chain saturated fatty acids, long chain unsaturated fatty acids, fatty acid esters, fatty acid ethers, witepsol, solid lipids, methyl stearate, triglycerides, glyceryl monostearate, glyceryl palmitostearate, stearic acid, palmitic acid, decanoic acid, behenic acid, beeswax, carnauba wax, paraffin, fatty acid triglycerides, fatty acid alcohols, PEG-fatty acid esters (with hydrophilic-lipophilic balance (HLB) below 13), PEG-surfactants with an HLB below 13, or combinations thereof.

In some aspects, the coating layer includes one or more excipients. Suitable excipients that can be used in some aspects of the present disclosure include, without limitation, organic and inorganic pharmaceutical excipients, natural products and derivatives thereof (such as sugars, vitamins, amino acids, peptides, proteins, and fatty acids), surfactants (anionic, cationic, non-ionic, and ionic), and mixtures thereof. The following list of excipients useful in the present disclosure is provided for exemplary purposes only and is not intended to be comprehensive. Many other excipients may be useful for purposes of the present disclosure, such as polyglutamic acid, polyacrilic acid, hyaluronic acid, alginate, PVA, PVP, Pluronic (PEO-PPO-PEO), cellulose, CMC, HPC, starch, chitosan, human serum albumin (HSA), phospholipids, fatty acid, fatty acid esters, triglycerides, beeswax, cyclodextrin, polysorbates, polyethylene glycol, polyvinylpyrrolidone (PVP) and aliphatic polyesters.

In some aspects, the excipients may feature a drug affinity part. The excipients of the present disclosure may feature a hydrophilic part. As is understood in the art, the terms “hydrophilic” and “hydrophobic” are relative terms. To function as an excipient in some aspects of the present disclosure, the excipient is a compound that includes polar or charged hydrophilic moieties as well as non-polar hydrophobic (lipophilic) moieties. The hydrophilic part can accelerate diffusion and increase permeation of the therapeutic agent into tissue. The hydrophilic part of the excipient may facilitate rapid movement of therapeutic agent off the expandable medical device during deployment at the target site by preventing hydrophobic drug molecules from clumping to each other and to the device, increasing drug solubility in interstitial spaces, and/or accelerating drug passage through polar head groups to the lipid bilayer of cell membranes of target tissues.

Exemplary excipients for application in the present disclosure may include chemical compounds with one or more hydroxyl, amino, carbonyl, carboxyl, acid, amide or ester moieties. Hydrophilic chemical compounds with one or more hydroxyl, amino, carbonyl, carboxyl, acid, amide or ester moieties having a molecular weight less than 5,000 to 10,000 are preferred in certain aspects. In other aspects, molecular weight of the excipient with one or more hydroxyl, amino, carbonyl, carboxyl, acid, amide, or ester moieties is preferably less than 1000 to 5,000, or more preferably less than 750 to 1,000, or most preferably less than 750. In these aspects, the molecular weight of the excipient is less than that of the therapeutic agent to be delivered.

In some aspects, the one or more excipients may be selected from amino alcohols, alcohols, amines, acids, amides and hydroxyl acids in both cyclo-and linear-aliphatic and aromatic groups. Examples include L-ascorbic acid and its salt, D-glucoascorbic acid and its salt, tromethamine, triethanolamine, diethanolamine, meglumine, glucamine, sodium docusate, urea, amine alcohols, glucoheptonic acid, glucomic acid, hydroxyl ketone, hydroxyl lactone, gluconolactone, glucoheptonolactone, glucooctanoic lactone, gulonic acid lactone, mannoic lactone, ribonic acid lactone, lactobionic acid, glucosamine, glutamic acid, benzyl alcohol, benzoic acid, hydroxybenzoic acid, propyl 4-hydroxybenzoate, lysine acetate salt, gentisic acid, lactobionic acid, lactitol, sorbitol, glucitol, sugar phosphates, glucopyranose phosphate, sugar sulphates, sugar alcohols, sinapic acid, vanillic acid, vanillin, methyl paraben, propyl paraben, xylitol, 2-ethoxyethanol, sugars, galactose, glucose, ribose, mannose, xylose, sucrose, lactose, maltose, arabinose, lyxose, fructose, cyclodextrin, (2-hydroxypropyl)-cyclodextrin, acetaminophen, ibuprofen, retinoic acid, lysine acetate, gentisic acid, catechin, catechin gallate, tiletamine, ketamine, propofol, lactic acids, acetic acid, salts of any organic acid and amine described above, polyglycidol, glycerol, multiglycerols, galactitol, di(ethylene glycol), tri(ethylene glycol), tetra(ethylene glycol), penta(ethylene glycol), di(propylene glycol), tri(propylene glycol), tetra(propylene glycol, and penta(propylene glycol), and combinations thereof. Some of the chemical compounds with one or more hydroxyl, amine, carbonyl, carboxyl, amide or ester moieties described herein are very stable under heating, survive an ethylene oxide sterilization process, and/or do not react with the therapeutic agent during sterilization.

In some aspects, the one or more excipients may be selected from amino acids and salts thereof. For example, the excipient may be one or more of alanine, arginine, asparagines, aspartic acid, cysteine, glutamic acid, glutamine, glycine, histidine, proline, isoleucine, leucine, lysine, methionine, phenylalanine, serine, threonine, tryptophan, tyrosine, valine, and derivatives thereof are. Certain amino acids, in their zwitterionic form and/or in a salt form with a monovalent or multivalent ion, have polar groups, relatively high octanol-water partition coefficients, and are useful in some facets of the present disclosure. In the context of the present disclosure “low-solubility amino acid” refers to amino acid having a solubility in unbuffered water of less than about 4% (40 mg/ml). These include cystine, tyrosine, tryptophan, leucine, isoleucine, phenylalanine, asparagine, aspartic acid, glutamic acid, and methionine.

Amino acid dimers, sugar-conjugates, and other derivatives may also be considered for excipients. Through simple reactions well known in the art hydrophilic molecules may be joined to hydrophobic amino acids, or hydrophobic molecules to hydrophilic amino acids, to make additional excipients useful in aspects of the present disclosure. Catecholamines, such as dopamine, levodopa, carbidopa, and DOPA, are also useful as excipients.

In some aspects, the excipient may be of a material that is at a glass transition temperature at 37 °C or higher. As identified herein, providing a material on the medical device that transitions to a sticky or tacky state in situ within the vessel of the subject allows for adhering the coating to the vessel wall. Such materials may include hydrogenated coconut oil, coconut oil, mineral oil, cetyl alcohol, petrolatum, petroleum jelly, decanol, soft paraffin, tridecanol, dodecanol, long chain saturated fatty acids, long chain unsaturated fatty acids, fatty acid esters, fatty acid ethers, witepsol, solid lipids, methyl stearate, triglycerides, glyceryl monostearate, glyceryl palmitostearate, stearic acid, palmitic acid, decanoic acid, behenic acid, beeswax, carnauba wax, paraffin, fatty acid triglycerides, fatty acid alcohols or combinations thereof.

In some aspects, the excipients may be liquid additives. One or more liquid excipients may be can be used in the medical device coating to improve the integrity of the coating. Without being bound by theory, a liquid excipient can improve the compatibility of the therapeutic agent in the coating mixture. The liquid excipients used in aspects of the present disclosure is not a solvent. The solvents such as ethanol, methanol, dimethylsulfoxide, and acetone, will be evaporated after the coating is dried. In other words, the solvent will not stay in the coating after the coating is dried. In contrast, the liquid excipients in aspects of the present disclosure will stay in the coating after the coating is dried. The liquid excipient is liquid or semi-liquid at room temperature and one atmosphere pressure. The liquid excipient may form a gel at room temperature. In some aspects, the liquid excipient may be a non-ionic surfactant. Examples of liquid excipients include PEG-fatty acids and esters, PEG-oil transesterification products, polyglyceryl fatty acids and esters, Propylene glycol fatty acid esters, PEG sorbitan fatty acid esters, and PEG alkyl ethers as mentioned above. Some examples of a liquid excipient are Tween 80, Tween 81, Tween 20, Tween 40, Tween 60, Solutol HS 15, Cremophor RH40, and Cremophor EL&ELP.

In some aspects, the excipient may be a surfactant; a chemical compound with one or more hydroxyl, amine, carbonyl, carboxyl, amides or ester moieties; or both. Exemplary surfactants may be chosen from PEG fatty esters, PEG omega-3 fatty esters and alcohols, glycerol fatty esters, sorbitan fatty esters, PEG glyceryl fatty esters, PEG sorbitan fatty esters, sugar fatty esters, PEG sugar esters, Tween 20, Tween 40, Tween 60, p-isononylphenoxypolyglycidol, PEG laurate, PEG oleate, PEG stearate, PEG glyceryl laurate, PEG glyceryl oleate, PEG glyceryl stearate, polyglyceryl laurate, polyglyceryl oleate, polyglyceryl myristate, polyglyceryl palmitate, polyglyceryl-6 laurate, polyglyceryl-6 oleate, polyglyceryl-6 myristate, polyglyceryl-6 palmitate, polyglyceryl-10 laurate, polyglyceryl-10 oleate, polyglyceryl-10 myristate, polyglyceryl-10 palmitate, PEG sorbitan monolaurate, PEG sorbitan monolaurate, PEG sorbitan monooleate, PEG sorbitan stearate, PEG oleyl ether, PEG laurayl ether, Tween 20, Tween 40, Tween 60, Tween 80, octoxynol, monoxynol, tyloxapol, sucrose monopalmitate, sucrose monolaurate, decanoyl-N-methylglucamide, n-decyl-β-D-glucopyranoside, n-decyl-β-D-maltopyranoside, n-dodecyl β -D-glucopyranoside, n-dodecyl-β-D-maltoside, heptanoyl-N-methylglucamide, n-heptyl-β-D-glucopyranoside, n-heptyl-β-D-thioglucoside, n-hexyl-β-D-glucopyranoside, nonanoyl-N-methylglucamide, n-nonyl-β-D-glucopyranoside, octanoyl-N-methylglucamide, n-octyl-β-D-glucopyranoside, octyl-β-D-thioglucopyranoside and their derivatives. In some aspects, the excipients may include one of sodium docusate sorbitol, urea, BHT, BHA, PEG-sorbitan monolaureate, petrolatum, methyl stearate or a combination thereof.

In some aspects, one or more of a surfactant or a small water-soluble molecule (the chemical compounds with one or more hydroxyl, amine, carbonyl, carboxyl, amides or ester moieties) with the therapeutic agent are in certain cases superior to only utilizing the therapeutic agent and a single excipient. By incorporating the one or more additional excipients, the drug coating may have increased stability during transit and rapid drug release when pressed against tissues of the lumen wall at the target site of therapeutic intervention when compared to some formulations comprising the therapeutic agent and only one excipient. Furthermore, the miscibility and compatibility of the therapeutic agent with the excipient or the drug coating with the medical device, generally, is improved by the presence of the one or more additional excipients. For example, a surfactant may allow for improved coating uniformity and integrity.

In some aspects, the coating layer(s) may include multiple excipients, and one excipient is more hydrophilic than one or more of the other excipients. In another embodiment, the coating layer multiple excipients, and one excipient has a different structure from that of one or more of the other excipients. In yet another aspect, the coating layer includes multiple excipients. Some aspects of the present disclosure may include a mixture of at least two additional excipients, for example, a combination of one or more surfactants and one or more chemical compound with one or more hydroxyl, amine, carbonyl, carboxyl, amides or ester moieties. For example, therapeutic agents may bind to extremely water-soluble small molecules more poorly than surfactants, which can lead to suboptimal coating uniformity and integrity. Some surfactants may adhere so strongly to the therapeutic agents and the surface of the medical device that the therapeutic agent is not able to rapidly release from the surface of the medical device at the target site. On the other hand, some water-soluble small molecules (with one or more hydroxyl, amine, carbonyl, carboxyl, amides or ester moieties) adhere so poorly to the medical device that they release therapeutic agents before it reaches the target site, for example, into serum during the transit of a coated balloon catheter to the site targeted for intervention. By incorporating a mixture of multiple excipients, the coating layer may have improved properties over a formulation with only one excipient or no excipient.

In some aspects, the one or more additional excipients may include an antioxidant. An antioxidant is a molecule capable of slowing or preventing the oxidation of other molecules. Oxidation reactions can produce free radicals and/or peroxides, which start chain reactions and may cause degradation of therapeutic agents. Antioxidants terminate these chain reactions by removing free radicals and inhibiting oxidation of the active agent by being oxidized themselves. Antioxidants are used as the one or more additional excipients in certain aspects to prevent or slow the oxidation of the therapeutic agents in the coatings for medical devices. Antioxidants are a type of free radical scavengers. The antioxidant may be used alone or in combination with other additional excipients in certain aspects and may prevent degradation of the active therapeutic agent during sterilization or storage prior to use. Some representative examples of antioxidants that may be used in the drug coatings of the present disclosure include, without limitation, oligomeric or polymeric proanthocyanidins, polyphenols, polyphosphates, polyazomethine, high sulfate agar oligomers, chitooligosaccharides obtained by partial chitosan hydrolysis, polyfunctional oligomeric thioethers with sterically hindered phenols, hindered amines such as, without limitation, p-phenylene diamine, trimethyl dihydroquinolones, and alkylated diphenyl amines, substituted phenolic compounds with one or more bulky functional groups (hindered phenols) such as tertiary butyl, arylamines, phosphites, hydroxylamines, and benzofuranones. Also, aromatic amines such as p-phenylenediamine, diphenylamine, and N, N′ disubstituted p-phenylene diamines may be utilized as free radical scavengers. Other examples include, without limitation, butylated hydroxytoluene (“BHT”), butylated hydroxyanisole (“BHA”), L-ascorbate (Vitamin C), Vitamin E, herbal rosemary, sage extracts, glutathione, resveratrol, ethoxyquin, rosmanol, isorosmanol, rosmaridiphenol, propyl gallate, gallic acid, caffeic acid, p-coumeric acid, p-hydroxy benzoic acid, astaxanthin, ferulic acid, dehydrozingerone, chlorogenic acid, ellagic acid, propyl paraben, sinapic acid, daidzin, glycitin, genistin, daidzein, glycitein, genistein, isoflavones, and tertbutylhydroquinone. Examples of some phosphites include di(stearyl)pentaerythritol diphosphite, tris(2,4-di-tert. butyl phenyl)phosphite, dilauryl thiodipropionate and bis(2,4-di-tert. butyl phenyl)pentaerythritol diphosphite. Some examples, without limitation, of hindered phenols include octadecyl-3,5,di-tert. butyl-4-hydroxy cinnamate, tetrakis-methylene-3-(3′,5′-di-tert. butyl-4-hydroxyphenyl)propionate methane 2,5-di-tert-butylhydroquinone, ionol, pyrogallol, retinol, and octadecyl-3-(3,5-di-tert. butyl-4-hydroxyphenyl)propionate. An antioxidant may include glutathione, lipoic acid, melatonin, tocopherols, tocotrienols, thiols, Beta-carotene, retinoic acid, cryptoxanthin, 2,6-di-tert-butylphenol, propyl gallate, catechin, catechin gallate, and quercetin. Preferable antioxidants are butylated hydroxytoluene (BHT) and butylated hydroxyanisole (BHA).

### EXAMPLES

A folded unexpanded balloon is prepared with a PTFE or ePTFE protective coating wrapped therearound with a minimal overlap to identify the distance in overlap required to allow for a coating layer on the balloon to be exposed following inflation. The PTFE or ePTFE is of a higher elastic modulus such that the wrapped protective layer will not expand as significantly as the underlying balloon, allowing the external surface of the balloon to become exposed as the balloon inflates.

Studies will be arranged with different types of PTFE and varying levels of overlap or negative overlap over a drug coated balloon, likely with paclitaxel, rapamycin, or a sirolimus drug. Balloons will then be inflated in the femoral arteries of porcine subjects and the arteries then examined at various time points for lumen area, lumen wall thickness and percentage of stenosis. Results will determine the best amount of overlap or exposed area of the drug covered layer that allows the protective layer to function while still providing the maximal amount of payload.

Additional studies will utilize a longer route of administration to determine the level of effectiveness over prolonged distance on both drug delivery and overall lumen health following expansion of the balloon.

While particular aspects have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

It is appreciated that all reagents are obtainable by sources known in the art unless otherwise specified.

It is also to be understood that this disclosure is not limited to the specific aspects and methods described herein, as specific components and/or conditions may, of course, vary. Furthermore, the terminology used herein is used only for the purpose of describing particular aspects of the present disclosure and is not intended to be limiting in any way. It will be also understood that, although the terms “first,” “second,” “third” etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, “a first element,” “component,” “region,” “layer,” or “section” discussed below could be termed a second (or other) element, component, region, layer, or section without departing from the teachings herein. Similarly, as used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Reference is made in detail to exemplary compositions, aspects and methods of the present disclosure, which constitute the best modes of practicing the disclosure presently known to the inventors. The Figures are not necessarily to scale. However, it is to be understood that the disclosed aspects are merely exemplary of the disclosure that may be embodied in various and alternative forms. Therefore, specific details disclosed herein are not to be interpreted as limiting, but merely as a representative basis for any aspect of the disclosure and/or as a representative basis for teaching one skilled in the art to variously employ the present disclosure.

Patents, publications, and applications mentioned in the specification are indicative of the levels of those skilled in the art to which the disclosure pertains. These patents, publications, and applications are incorporated herein by reference to the same extent as if each individual patent, publication, or application was specifically and individually incorporated herein by reference.

The foregoing description is illustrative of particular embodiments of the disclosure, but is not meant to be a limitation upon the practice thereof. The following claims, including all equivalents thereof, are intended to define the scope of the disclosure.

1. A balloon catheter for delivering a therapeutic agent to a blood vessel, the balloon catheter comprising:
an elongate member having a lumen and a distal end;
an expandable balloon attached to the distal end of the elongate member and in fluid communication with the lumen;
a coating layer overlying an exterior surface of the expandable balloon, the coating layer comprising a therapeutic agent or a polymer microparticle containing the therapeutic agent and an excipient; and
a protective layer overlying all or at least 75% of the coating layer when the expandable balloon in a folded, deflated position, wherein the protective layer is of a material with higher elastic modulus than the expandable balloon.

2. The balloon catheter of claim 1, wherein the protective layer comprises at least one band about the circumference of the expandable balloon.

3. The balloon catheter of claim 2, wherein the protective layer comprises at least two bands about the circumference of the expandable balloon.

4. The balloon catheter of claim 3, wherein each band is separated by a distance with exposed coating layer therebetween.

5. The balloon catheter of claim 3, wherein the bands are positioned in an overlap, wherein the overlap is sufficient to allow the coating layer to be exposed to a vessel wall when the balloon is expanded therein.

6. The balloon catheter of claim 1, wherein the protective layer is a ribbon connected to a distal end and a proximal end of the expandable balloon and further wherein the ribbon wraps around the length of the balloon between the distal and proximal ends.

7. The balloon catheter of claim 6, wherein the ribbon wraps at least once around the circumference of the balloon.

8. The balloon catheter of claim 7, wherein the ribbon is wrapped such that the ribbon overlaps with itself as it progresses along the length of the expandable balloon.

9. The balloon catheter of claim 1, wherein the protective layer has a modulus of elasticity of from 0.3 to about 2.5 GPa.

10. The balloon catheter of claim 1, wherein the protective layer is comprised of polytetrafluoroethylene (PTFE), expanded polytetrafluorethylene (ePTFE), chlorotrifluoroethylene (E-CTFE), perfluoroalkoxy (PFA), or ethylene tetrafluoroethylene (ETFE).

11. The balloon catheter of claim 1, wherein the protective layer has a thickness of from 100 μm to 600 μm.

12. The balloon catheter of claim 1, wherein the thickness of the protective layer varies.

13. The balloon catheter of claim 1, wherein the coating layer comprises a therapeutic agent.

14. The balloon catheter of claim 13, wherein the therapeutic agent is chosen from paclitaxel, rapamycin, daunorubicin, 5-fluorouracil, doxorubicin, sunitinib, sorafenib, irinotecan, bevasizumab, cetuxamab, biolimus (biolimus A9), everolimus, zotarolimus, tacrolimus, dexamethasone, prednisolone, corticosterone, cisplatin, vinblastine, lidocaine, bupivacaine, bosutinib, ceritinib, crizotinib, gefitinib, ruxolitinib, imatinib, axitinib, nilotinib, trametinib, afatinib, ibrutinib, cabozantinib, imatinib, lenvatinib, sunitinib, regorafenib, sorafenib, vandetanib, dasatinib, pazopanib, triamciclone, tranilast, halofuginone, montelukast, zafirlukast, pirfenidone, Y27632, CA3, verteporfin, VGLL4 peptide, nintedanib, avapritinib, abemaciclib, erdafitinib, fedratinib, nilotinib, nintendanib, palbociclib, pemigatinib, xanthines, aminophylline, sildenafil, tadalafil, vardenafil, udenafil, avanafil, dipyridamole, quinazoline, paraxanthine, papaverine, mesembrenone, rolipram, ibudilast, piclamilast, luteolin, drotaverine, roflumilast, apremilast, crisaborole, inamrinone, milrinone, enoximone, anagrelide, cilostazol, pimobendan, erythro-9-(2-hydroxy-3-nonyl)adenine), (2-[(3,4-dimethoxyphenyl)methyl]-7-[(1R)-1-hydroxyethyl]-4-phenylbutyl]-5-methyl-imidazo[5,1-f][1,2,4]triazin-4(1H)-one), oxindole, (9-(6-phenyl-2-oxohex-3-yl)-2-(3,4-dimethoxybenzyl)-purin-6-one), 3-isobutyl-1-methylxanthine, pentoxifylline, theobromine, theophylline, resveratrol, quercetin, curcumin, chrysin, myricetin, luteolin, apigenin, anthrocyanin, genistein, epigallocatechin gallate, fisetin, astxanthin, tetrahydrocurcumin, imatinib, nintedanib, sorafenib, sunitinib, pazopanib, ROCK inhibitor (Y27632), YAP/TAZ inhibitor (CA3 and verteporfin), YAP/TAZ-TEAD interaction inhibitor (verteporfin, VGLL4 peptide), SRC inhibitor (dasatinib), and combinations thereof.

15. The balloon catheter of claim 13, wherein the coating layer further comprises a biodegradable polymer.

16. The balloon catheter of claim 15, wherein the biodegradable polymer is chosen from a polylactic acid polymer, polycaprolactone (PCL), poly lactic-co-glycolic acid (PLGA), and poly(ethylene glycol) methyl ether-block-poly(lactide-co-glycolide) (PLGA-b-mPEG).

17. The balloon catheter of claim 13, wherein the coating layer further comprises an excipient chosen from a fatty acid, a fatty acid ester, polylactic acid (PLLA, PDLA, PDLLA), polycaprolactone (PCL), sodium docusate, PLGA, PLGA-b-mPEG, polyglutamic acid, polyacrilic acid, hyaluronic acid, alginate, PVA, PVP, Pluronic (PEO-PPO-PEO), cellulose, CMC, HPC, starch, chitosan, human serum albumin (HSA), phospholipids, fatty acid, fatty acid esters, triglycerides, beeswax, cyclodextrin, Tween 20, Tween 80, TPGS, SLS, butylated hydroxytoluene, vitamin E, vitamin E succinate, tannic acid, polyethylene glycol, N-isopropylacrylamide, and sorbitol esters.

18. The balloon catheter of claim 13, wherein the coating layer further comprises a hydrophobic material containing the therapeutic agent embedded therein.

19. The balloon catheter of claim 18, wherein the hydrophobic material comprises a hydrophobic material with a glass transition temperature of 37 °C or lower.

20.-30 (canceled)

31. A balloon catheter for delivering a therapeutic agent to a blood vessel, the balloon catheter comprising:
an elongate member having a lumen and a distal end;
an expandable balloon attached to the distal end of the elongate member and in fluid communication with the lumen;
a porous layer overlying an exterior surface of the expandable balloon, the porous layer comprising
a therapeutic agent or a polymer microparticle containing the therapeutic agent and an excipient; and
a protective layer overlying all or at least 50% of the coating layer when the expandable balloon in a folded, deflated position, wherein the protective layer is of a material with higher elastic modulus than the expandable balloon.

32-35. (canceled)
